(19) United States
(12) Patent Application Publication
(10) Pub. No.: US 2024/0219376 A1
(43) Pub. Date: Jul. 4, 2024

(54) SYSTEMS AND METHODS FOR LABEL-FREE SENSING OF NEUTROPHIL ACTIVATION

(71) Applicant: Wisconsin Alumni Research Foundation, Madison, WI (US)

(72) Inventors: Melissa C. Skala, Middleton, WI (US); Rupsa Datta, Madison, WI (US); Alexandra Jule Walsh, Madison, WI (US)

(21) Appl. No.: 18/403,499

(22) Filed: Jan. 3, 2024

Related U.S. Application Data
(60) Provisional application No. 63/437,092, filed on Jan. 4, 2023.

(51) Int. Cl. G01N 33/50; G01N 15/14; G16B 5/20

(52) U.S. Cl. CPC G01N 33/5091; G01N 15/14; G16B 5/20

(57) ABSTRACT

The present disclosure provides a new sensing device and method for determining the activation status of neutrophils. The device has an autofluorescence spectrometer and a cell analysis observation zone. A current activation status of the neutrophil cell is identified based on a current activation prediction, where the current activation prediction is computed using at least a portion of the autofluorescence data set. The current activation prediction is computed using at least two metabolic endpoints of the autofluorescence data set and at least one morphological parameter as an input. The at least two metabolic endpoints include either: reduced nicotinamide adenine dinucleotide and/or reduced nicotinamide dinucleotide phosphate (NAD(P)H) mean fluorescence lifetime (τₘ), NAD(P)H shortest fluorescence amplitude component (α₁), and NAD(P)H shortest fluorescence lifetime component (τ₁); or the NAD(P)H τₘ and flavin adenine dinucleotide (FAD) τₘ. The at least one morphological parameter includes either the solidity or the eccentricity.

### CROSS-REFERENCE TO RELATED APPLICATIONS

This application is related to, claims priority to, and incorporates herein by reference for all purposes U.S. Provisional Patent Application No. 63/437,092, filed Jan. 4, 2023.

### STATEMENT REGARDING FEDERALLY FUNDED RESEARCH

This invention was made with government support under CA205101 awarded by the National Institutes of Health. The government has certain rights in the invention.

### BACKGROUND

Neutrophils or polymorphonuclear (PMN) leukocytes are the most abundant white blood cell (50-70%) in human peripheral blood. These cells are an essential component of the innate immune system, and the first to reach sites of infection and inflammation. Circulating neutrophils are quiescent, and their activation is a critical step in a healthy immune response. Upon activation, they combat invading pathogens by diverse effector functions like the oxidative burst, phagocytosis, and neutrophil extracellular traps (NETs). Neutrophils also respond to multiple signals by secreting several cytokines and other inflammatory factors to regulate inflammation as well as the immune system. Recent studies have revealed additional functions of neutrophils beyond resolution of inflammation including modulating neighboring cells and priming macrophages for long-term immune response. Neutrophils play prominent roles in diseases like cancer and autoimmune disorders. In fact, there is accumulation of neutrophils in the peripheral blood of cancer patients, especially at advanced stages. Due to the critical role of neutrophils in host defense and other human diseases, their presence and activation are important markers of inflammation, human immune health, and response to therapy. Assessment of neutrophils is also critical for neutrophil adoptive cell therapy in cancer or for immune profiling ex vivo.

Current techniques to evaluate neutrophil activation and function are destructive or provide bulk population level information. These include single-cell measurements with high-throughput flow cytometry using fluorescent antibodies, functional measurements using fluorescent dyes, bulk measurements of oxygen consumption rate to detect oxidative burst, or bulk cytokine release assays. Single-cell RNA sequencing (scRNA-seq) also provides single-cell measurements, however there is a scarcity of published data on neutrophils. Thus, there is a need for a label-free, non-destructive method to evaluate neutrophil activation and function.

Given the relevance of neutrophils for inflammation, cancer, and infectious disease, new label-free and non-destructive tools are needed to assess neutrophil activation status in single cells.

### SUMMARY

The present disclosure provides a new sensing device for determining the activation status of neutrophils. The neutrophil activation status sensing device has a cell analysis observation zone adapted to receive a neutrophil cell and to present the neutrophil cell for individual autofluorescence interrogation. The activation sensing device has an autofluorescence spectrometer configured to acquire an autofluorescence data set for the neutrophil cell located in the cell analysis observation zone. The autofluorescence spectrometer has a light source, a photon-counting detector, photon-counting electronics, and a processor in electronic communication with the autofluorescence spectrometer. The autofluorescence spectrometer has a non-transitory computer-readable medium accessible to the processor and has stored instructions that, when executed by the processor, cause the processor to: a) receive the autofluorescence data set, and b) identify a current activation status of the neutrophil cell based on a current activation prediction. The current activation prediction is computed using at least a portion of the autofluorescence data set. The current activation prediction is computed using at least two metabolic endpoints of the autofluorescence data set and at least one morphological parameter as an input, where the at least two metabolic endpoints include either: reduced nicotinamide adenine dinucleotide and/or reduced nicotinamide dinucleotide phosphate (NAD(P)H) mean fluorescence lifetime (τₘ), NAD(P)H shortest fluorescence amplitude component (α₁), and NAD(P)H shortest fluorescence lifetime component (τ₁); or the NAD(P)H τₘ and flavin adenine dinucleotide (FAD) τₘ, and the at least one morphological parameter includes either solidity or eccentricity.

In another aspect, the present disclosure provides a method of characterizing neutrophil activation status. The method includes the following steps: a) optionally receiving a population of neutrophil cells having unknown activation status, b) acquiring an autofluorescence data set from a neutrophil cell of the population of neutrophil cells, and c) identifying a current activation status of the neutrophil cell based on a current activation prediction. The current activation prediction is computed using at least a portion of the autofluorescence data set, and the current activation prediction is computed using at least two metabolic endpoints of the autofluorescence data set and at least one morphological parameter as an input. The at least two metabolic endpoints include either: reduced nicotinamide adenine dinucleotide and/or reduced nicotinamide dinucleotide phosphate (NAD(P)H) mean fluorescence lifetime (τₘ), NAD(P)H shortest fluorescence amplitude component (α₁), and NAD(P)H shortest fluorescence lifetime component (τ₁); the NAD(P)H τₘ and flavin adenine dinucleotide (FAD) τₘ, and the at least one morphological parameter includes either solidity or eccentricity.

In a further aspect, the present disclosure provides a method of classifying neutrophil activation status. The method includes the following steps: a) receiving a population of neutrophil cells having unknown activation status; b) acquiring an autofluorescence data set for each neutrophil cell of the population of neutrophil cells, each autofluorescence data set including autofluorescence lifetime information; and either: c1) physically isolating a first portion of the population of neutrophil cells from a second portion of the population of neutrophil cells based on a current activation prediction, wherein each neutrophil cell of the population of neutrophil cells is placed into the first portion when the current activation prediction exceeds a predetermined threshold and into the second portion when the current activation prediction is less than or equal to the predetermined threshold; or c2) generating a report including the current activation prediction, the report optionally identifying a proportion of the population of neutrophil cells having the current activation prediction that exceeds the predetermined threshold. The current activation prediction is computed using at least two metabolic endpoints of the autofluorescence data set and at least one morphological parameter as an input. The at least two metabolic endpoints include either: reduced nicotinamide adenine dinucleotide and/or reduced nicotinamide dinucleotide phosphate (NAD(P)H) mean fluorescence lifetime (τₘ), NAD(P)H shortest fluorescence amplitude component (α₁), and NAD(P)H shortest fluorescence lifetime component (τ₁); or the NAD(P)H τₘ and flavin adenine dinucleotide (FAD) τₘ. The at least one morphological parameter includes either solidity or eccentricity.

### BRIEF DESCRIPTIONS OF THE DRAWINGS

FIG. 1 is a flowchart illustrating a method, in accordance with an aspect of the present disclosure.

FIG. 2 is a flowchart illustrating a method, in accordance with an aspect of the present disclosure.

FIG. 3 is a flowchart illustrating a method, in accordance with an aspect of the present disclosure.

FIG. 4 is a block diagram of a device, in accordance with an aspect of the present disclosure.

FIG. 5 shows results using the NAD(P)H τₘ, NAD(P)H α₁, NAD(P)H α₂, NAD(P)H τ₁, NAD(P)H τ₂, FAD τ₁, and FAD τₘ as metabolic endpoints and solidity, eccentricity, and major axis length, and convex area as morphological parameters, where the morphological parameters were computed from the NADH image.

FIG. 6 shows results using the NAD(P)H τₘ, NAD(P)H α₁, and NAD(P)H shortest fluorescence lifetime component (τ₁) as metabolic endpoints and solidity and eccentricity as morphological parameters, where the morphological parameters were computed from the NADH image.

FIG. 7 shows results using the NAD(P)H τₘ and the FAD τₘ as metabolic endpoints and solidity and eccentricity as morphological parameters, where the morphological parameters were computed from the NADH image.

FIG. 8 shows results using the NAD(P)H τₘ and the FAD τₘ as metabolic endpoints and eccentricity as a morphological parameter, where the morphological parameter was computed from the NADH image.

FIG. 9 shows in vivo metabolic imaging upon neutrophil activation using NAD(P)H lifetime and FAD lifetime, as well as mCherry-labeled neutrophils in transgenic zebrafish.

### DETAILED DESCRIPTION

Before the present invention is described in further detail, it is to be understood that the invention is not limited to the particular embodiments described. It is also understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices and methods relating to modifying biological molecules are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, the term “FAD” refers to flavin adenine dinucleotide.

As used herein, the term “memory” includes a non-volatile medium, e.g., a magnetic media or hard disk, optical storage, or flash memory; a volatile medium, such as system memory, e.g., random access memory (RAM) such as DRAM, SRAM, EDO RAM, RAMBUS RAM, DR DRAM, etc.; or an installation medium, such as software media, e.g., a CD-ROM, or floppy disks, on which programs may be stored and/or data communications may be buffered. The term “memory” may also include other types of memory or combinations thereof.

As used herein, the term “NAD(P)H” refers to reduced nicotinamide adenine dinucleotide and/or reduced nicotinamide dinucleotide phosphate.

As used herein, the term “neutrophil” refers to an immune cell type that is differentiated from a myeloblast, which itself is differentiated from a granulocyte-monocyte progenitor cells (GMPs) which itself is differentiated from a lymphoid-primed multipotent progenitors (LMPPs), which itself is derived from a multipotential hematopoietic stem cell.

As used herein, “nuclear parameter” refers to a measured geometric or clustering property of a nucleus of a cell of interest as determined by analyzing an acquired image of the cell of interest.

As used herein, “morphological parameter” refers to a solidity, eccentricity, an area of the neutrophil cell, a perimeter of the neutrophil cell, convex area which is the area of the convex hull (i.e., the smallest convex polygon that fits around the cell) that encloses a neutrophil cell, major axis length (longest line that can be drawn through the cell) or a combination thereof. The morphological parameter of solidity is defined as the ratio of the cell area to the convex hull of the cell. The morphological parameter of eccentricity is the ratio of the length of the minor axis length to the length of the major axis of the cell. The minor axis length is the longest line that can be drawn through the cell while remaining perpendicular with the major axis of the cell. Eccentricity is a measure of roundness of the cell and is sometimes referred to as ellipticity.

As used herein, the term “processor” may include one or more processors and memories and/or one or more programmable hardware elements. As used herein, the term “processor” is intended to include any of types of processors, CPUs, GPUs, microcontrollers, digital signal processors, or other devices capable of executing software instructions.

As used herein, the term “redox ratio” or “optical redox ratio” refers to a ratio of NAD(P)H fluorescence intensity to FAD fluorescence intensity; a ratio of FAD fluorescence intensity to NAD(P)H fluorescence intensity; a ratio of NAD(P)H fluorescence intensity to any arithmetic combination including FAD fluorescence intensity; or a ratio of FAD fluorescence intensity to any arithmetic combination including NAD(P)H fluorescence intensity. In certain cases, the redox ratio or optical redox ratio refers to a ratio of NAD(P)H fluorescence intensity to the sum of NAD(P)H and FAD fluorescence intensity.

Autofluorescence endpoints include photon counts/intensity and fluorescence lifetimes. The fluorescence lifetime of cells can be a single value, the mean fluorescence lifetime, or compromised from the lifetime values of multiple subspecies with different lifetimes. In this case, multiple lifetimes and lifetime component amplitude values are extracted from an autofluorescence data set. Both NAD(P)H and FAD can exist in quenched (short lifetime) and unquenched (long lifetime) configurations; therefore, the fluorescence decays of NAD(P)H and FAD are fit to two components. Generally, NADH and FAD fluorescence lifetime decays are fit to a two component exponential decay, I(t)=α₁ₑ^{-t/τ1}+α₂ₑ^{-t/τ2}+C, where I(t) is the fluorescence intensity as a function of time, t, after the laser pulse, α₁ and α₂ are the fractional contributions of the short and long lifetime components, respectively (i.e., α₁+α₂=1), τ₁ and τ₂ are the short and long lifetime components, respectively, and C accounts for background light. However, the lifetime decay can be fit to more components (in theory any number of components, although practically up to ˜5-6) which would allow quantification of additional lifetimes and component amplitudes. By convention lifetimes and amplitudes are numbered from short to long, but this could be reversed. A mean lifetime can be computed from the lifetime components, (τₘ=α₁τ₁+α₂τ₂ . . . ). Fluorescence lifetimes and lifetime component amplitudes can also be approximated from frequency domain data collection and analysis, time-correlated single-photon counting or phasor analysis, and gated cameras/detectors. For gated detection, α₁ could be approximated by dividing the detected intensity at early time bins by later time bins. Alternatively, fluorescence anisotropy can be measured by polarization-sensitive detection of the autofluorescence, thus identifying free NAD(P)H as the short rotational diffusion time in the range of 100-700 ps.

FAD α₁ refers to the contribution of bound FAD and is the shortest lifetime that is not dominated (i.e., greater than 50%) by instrument response and/or scattering. FAD α₁ is the contribution associated with FAD lifetime values from 50-1500 ps, from 50-1000 ps, or from 50-600 ps. For clarity, a claim herein including features related to a “shortest” lifetime cannot be avoided by defining the lifetime values to include a sacrificial shortest lifetime that is dominated by instrument response and/or scattering.

FAD τ₁ refers to the bound FAD lifetime and is the shortest lifetime that is not dominated (i.e., greater than 50%) by instrument response and/or scattering. FAD τ₁ is the FAD lifetime values from 50-1500 ps, from 50-1000 ps, or from 50-600 ps. For clarity, a claim herein including features related to a “shortest” lifetime cannot be avoided by defining the lifetime values to include a sacrificial shortest lifetime that is dominated by instrument response and/or scattering.

FAD 12 refers to the free FAD lifetime and is the longest lifetime that is not dominated (i.e., greater than 50%) by instrument response and/or scattering. FAD 12 is the FAD lifetime values from 1000-4000 ps, from 1000-3000 ps, or from 1500-3000 ps. For clarity, a claim herein including features related to a “longest” lifetime cannot be avoided by defining the lifetime values to include a sacrificial shortest lifetime that is dominated by instrument response and/or scattering.

$$FAD = {τ}_{m} = {α}_{1} \cdot {τ}_{1} + ( 1 - {α}_{1} ) \cdot {τ}_{2}$$

NAD(P)H α₁ refers to the contribution of free NAD(P)H and is the shortest lifetime that is not dominated (i.e., greater than 50%) by instrument response and/or scattering. NAD(P)H α₁ is the contribution associated with NAD(P)H lifetime values from 40-1500 ps, from 50-1000 ps, or from 50-600 ps. For clarity, a claim herein including features related to a “shortest” lifetime cannot be avoided by defining the lifetime values to include a sacrificial shortest lifetime that is dominated by instrument response and/or scattering.

NAD(P)H τ₁ refers to the free NAD(P)H lifetime and is the shortest lifetime that is not dominated (i.e., greater than 50%) by instrument response and/or scattering. NAD(P)H τ₁ is the NAD(P)H lifetime values from 200-1500 ns, from 200-1000 ns, or from 200-600 ns. For clarity, a claim herein including features related to a “shortest” lifetime cannot be avoided by defining the lifetime values to include a sacrificial shortest lifetime that is dominated by instrument response and/or scattering.

NAD(P)H τ₂ refers to the bound NAD(P)H lifetime and is the longest lifetime that is not dominated (i.e., greater than 50%) by instrument response and/or scattering. NAD(P)H τ₂ is the NAD(P)H lifetime values from 1000-4000 ns, from 1000-3000 ns, or from 1500-3000 ns. For clarity, a claim herein including features related to a “longest” lifetime cannot be avoided by defining the lifetime values to include a sacrificial shortest lifetime that is dominated by instrument response and/or scattering.

$$NAD ( P ) H {τ}_{m} = {α}_{1} \cdot {τ}_{1} + ( 1 - {α}_{1} ) \cdot {τ}_{2}$$

The various aspects may be described herein in terms of various functional components and processing steps. It should be appreciated that such components and steps may be realized by any number of hardware components configured to perform the specified functions.

### Methods

This disclosure provides a variety of methods. It should be appreciated that various methods are suitable for use with other methods. Similarly, it should be appreciated that various methods are suitable for use with the systems described elsewhere herein. When a feature of the present disclosure is described with respect to a given method, that feature is also expressly contemplated as being useful for the other methods and systems described herein, unless the context clearly dictates otherwise.

The methods described herein include predictions regarding the activation status of a given neutrophil cell. This prediction is a current activation prediction, which provides a computer-generated prediction for the current state of activation in a given neutrophil cell of interest. For example, the current activation prediction may indicate that a given neutrophil cell is activated or it may indicate that the cell is unstimulated.

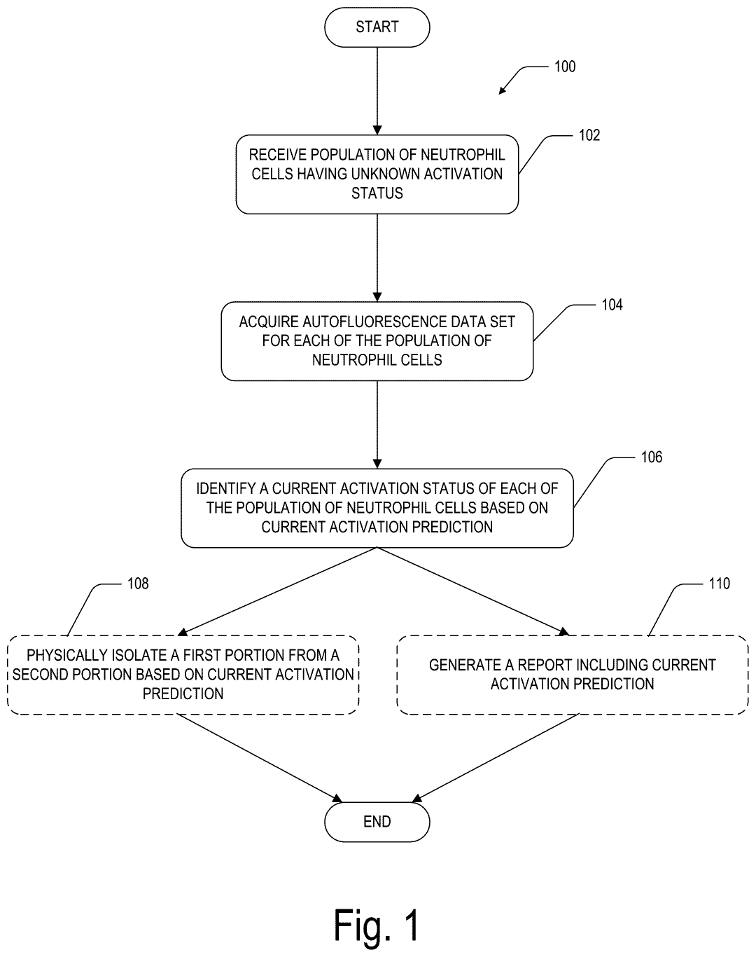

Referring to FIG. 1, the present disclosure provides a method 100 of characterizing neutrophil activation status. At process block 102, the method 100 optionally includes receiving a population of neutrophil cells having unknown activation status. The population of neutrophil cells can itself be contained within a broader population of cells that includes some cells that are not neutrophil cells. At process block 104, the method 100 includes acquiring an autofluorescence data set for each neutrophil cell of the population of neutrophil cells. At process block 106, the method 100 includes identifying a current activation status of each of the neutrophil cells based on a current activation prediction. The current activation prediction is computed using at least a portion of the autofluorescence data set. The current activation prediction is computed using at least two metabolic endpoints of the autofluorescence data set and at least one morphological parameter as an input. The at least one metabolic endpoint includes those described below. Following process block 106, the method 100 can proceed to process block 108 or 110, depending on the desired outcome. In some cases, the method 100 proceeds to process block 108 and process block 110, in either order. While process blocks 108 and 110 are both illustrated and described as optional, the method 100 includes either process block 108 or process block 110. At optional process block 108, the method 100 optionally includes physically isolating a first portion of the population of neutrophil cells from a second portion of the population of neutrophil cells based on a current activation prediction, wherein each neutrophil cell of the population of neutrophil cells is placed into the first portion when the current activation prediction exceeds a predetermined threshold and into the second portion when the current activation prediction is less than or equal to the predetermined threshold. At optional process block 110, the method 100 optionally includes generating a report including the current activation prediction. The report optionally includes identifying a proportion of the population of neutrophil cells having a current activation prediction that exceeds a predetermined threshold.

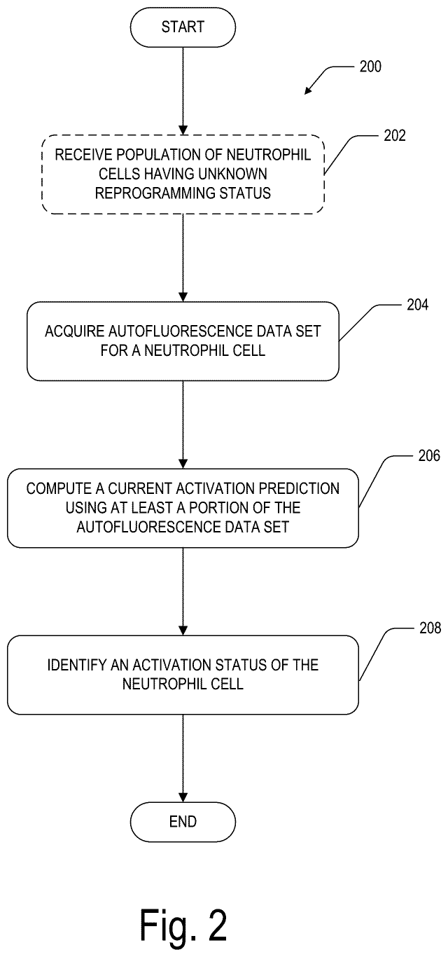

Referring to FIG. 2, the present disclosure provides a method 200 of characterizing neutrophil cell activation status. At optional process block 202, the method 200 optionally includes receiving a population of neutrophil cells having unknown activation status. At process block 204, the method 200 includes acquiring an autofluorescence data set from a neutrophil cell of the population of neutrophil cells. At process block 206, the method 200 includes computing a current activation prediction using at least a portion of the autofluorescence data set. The current activation prediction is computed using at least one metabolic endpoint and optionally using at least one morphological parameter. The at least one metabolic endpoint includes those outlined below. At process block 208, the method 200 includes identifying a current activation status of the neutrophil cell based on the current activation prediction.

Method 100 and method 200 are related to one another and can be utilized together. For example, method 200 can be utilized within method 100. Aspects described with respect to method 100 can be utilized in method 200, unless the context clearly dictates otherwise, and vice versa.

The autofluorescence data set acquired at process block 104 or 204 can be acquired in a variety of ways, as would be understood by one having ordinary skill in the spectroscopic arts with knowledge of this disclosure and their own knowledge from the field. For example, the autofluorescence data can be acquired from fluorescence decay data. As another example, the autofluorescence data can be acquired by gating a detector (a camera, for instance) to acquire data at specific times throughout a decay in order to approximate the autofluorescence endpoints described herein. As yet another example, a frequency domain approach can be used to measure and analyze lifetime. Alternatively, fluorescence anisotropy can be measured by polarization-sensitive detection of the autofluorescence, thus identifying free NAD(P)H as the short rotational diffusion time in the range of 100-700 ps. The specific way in which autofluorescence data is acquired is not intended to be limiting to the scope of the present invention, so long as the lifetime information necessary to determine the autofluorescence endpoints necessary for the methods described herein can be suitably measured, estimated, or determined in any fashion. One example of a suitable autofluorescence data set acquisition is described below in the Examples section.

The physical isolation operation of optional process block 108 is in response to a current activation prediction determined from the acquired autofluorescence data set. If the current activation prediction exceeds a predetermined threshold for a given neutrophil cell, then that neutrophil cell is placed into the first portion. If the current activation prediction is less than or equal to the predetermined threshold for the given neutrophil cell, then that neutrophil cell is placed into the second portion. The result of this physical isolation is that the first portion of the population of neutrophil cells is significantly enriched in neutrophil cells having a given activation status (e.g., activated or unstimulated), whereas the second portion of the population of neutrophil cells is significantly depleted of neutrophil cells having that given activation status.

In some cases, the physical isolation operation of optional process block 108 can include isolating cells into three, four, five, six, or more portions. In these cases, the different portions will be separated by a number of predetermined thresholds that is one less than the number of portions (i.e., three portions=two predetermined thresholds). The portion whose current activation prediction exceeds all of the predetermined thresholds (i.e., exceeds the highest threshold) contains the greatest concentration of neutrophil cells with a given activation status. The portion whose current activation prediction fails to exceed any of the predetermined thresholds (i.e., fails to exceed the lowest threshold) contains the lowest concentration of neutrophil cells with the given activation status. Using multiple predetermined thresholds can afford the preparation of portions of the population of neutrophil cells that have extremely high or extremely low concentrations of neutrophil cells with the given activation status. In some cases, the physical isolation operation of optional process block 108 (or a totally separate aspect of method 100, as would be appreciated by those having ordinary skill in the cell isolation arts) can include isolating other kinds of cells, such as red blood cells or the like, or various kinds of debris so they are not included in the portions including neutrophil cells.

The current activation prediction is computed using at least two metabolic endpoints of the autofluorescence data set and at least one whole cell morphological parameter for each neutrophil cell of the population of neutrophil cells as an input. The current activation prediction is computed using an equation or model that is generated by a machine learning process on data for a population of neutrophil cells having a known activation status using the at least one metabolic endpoint and optionally the at least one morphological parameter as a variable.

The at least one metabolic endpoint can include the NAD(P)H τₘ; NAD(P)H α₂; NAD(P)H α₁; FAD shortest fluorescence lifetime component τ₁; NAD(P)H τ₁; FAD mean fluorescence lifetime τₘ; NAD(P)H τ₂, or a combination thereof.

The at least one metabolic endpoint can also optionally includes the FAD shortest lifetime amplitude component (α₁), the FAD longest fluorescence lifetime component (12), or a combination thereof. The at least one metabolic endpoint can also optionally include one or more of the following: NAD(P)H fluorescence intensity; FAD fluorescence intensity; an optical redox ratio (i.e., NAD(P)H/[NAD(P)H+FAD], see definition above); NAD(P)H shortest lifetime amplitude component or NAD(P)H α₁; NAD(P)H mean fluorescence lifetime or NAD(P)H τₘ; NAD(P)H shortest fluorescence lifetime or NAD(P)H τ₁; NAD(P)H second shortest fluorescence lifetime or NAD(P)H τ₂. The morphological parameters can include solidity, eccentricity, major axis length, and convex area.

In some cases, two, three, four, five, six, seven, eight, nine, ten, eleven, twelve, thirteen, fourteen or more inputs are used.

In some cases, a surprisingly small number of metabolic endpoints and morphological parameters can be used while still achieving an adequate level of classification accuracy. In one instance, a good level of classification was achieved using only the NAD(P)H τₘ and the FAD τₘ as metabolic endpoints and eccentricity as a morphological parameter. An AUC of 0.90 was obtained in this instance, where AUC is the area under the receiver operating curve. AUC provides an aggregate measure of performance across all possible classification thresholds. One way of interpreting AUC is as the probability that the model ranks a random positive example more highly than a random negative example.

In another instance, a good level of classification was achieved using only the NAD(P)H τₘ and the FAD τₘ as metabolic endpoints and solidity and eccentricity as morphological parameters. An AUC of 0.91 was obtained in this instance.

In another instance, a good level of classification was achieved using only the NAD(P)H τₘ, NAD(P)H α₁, and NAD(P)H τ₁ as metabolic endpoints and eccentricity as a morphological parameter. An AUC of 0.92 was obtained in this instance.

In another instance, a good level of classification was achieved using only the NAD(P)H τₘ, NAD(P)H α₁, NAD(P)H α₂, NAD(P)H τ₁, NAD(P)H τ₂, FAD τ₁, and FAD τₘ as metabolic endpoints and solidity, eccentricity, and major axis length, and convex area as morphological parameters. An AUC of 0.96 was obtained in this instance.

In some cases, the predictions described herein are computed using a phasor analysis, as described in International Patent Application Pub. No. 2021/232011, which is incorporated herein in its entirety by reference. Briefly, a first phasor at a first frequency and a second phasor at a second, different frequency are computed from the time-resolved autofluorescence decay, and then the activation prediction can be computed using these phasors.

The method 100 or method 200 can sort neutrophil cells into activation categories based on the current activation status.

The method 100 or method 200 can provide surprising accuracy of classifying neutrophil cell current activation state. The accuracy can be at least 80%, at least 82.5%, at least 85%, at least 87.5%, at least 90%, at least 92.5%, at least 95%, or at least 96%. One non-limiting example of measuring the accuracy includes executing the method 100 or method 200 on a given cell with unknown current activation status and then using one of the traditional methods for determining activation status (which will typically be a destructive method) for a number of cells that is statistically significant.

The method 100 or method 200 can be performed without the use of a fluorescent label for binding the neutrophil cell. The method 100 or method 200 can be performed without immobilizing the neutrophil cell.

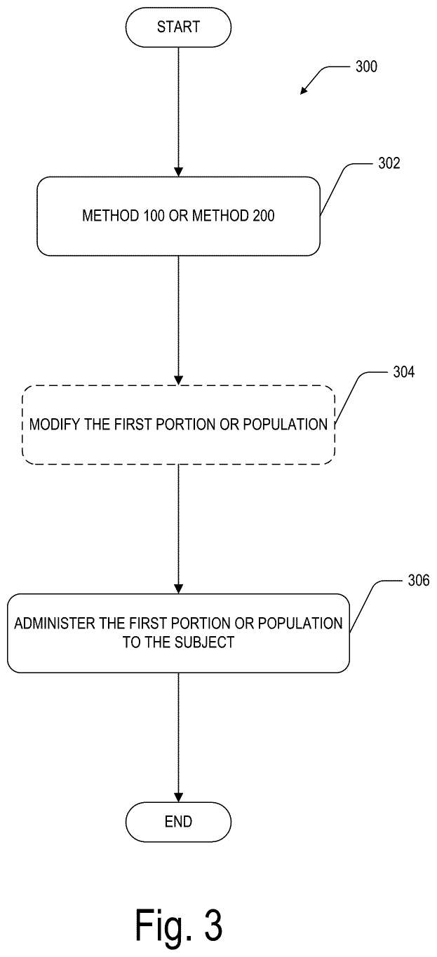

Referring to FIG. 3, the present disclosure provides a method 300 of administering activated neutrophil cells to a subject in need thereof. At process block 302, the method 300 includes the method 100 or method 200 described above, which results in a first portion of the population of neutrophil cells enriched for current activation state (when optional process block 108 is utilized) or results in a report identifying the proportion of neutrophil cells that have a given current activation state (when optional process block 110 is utilized). At optional process block 304, the method 300 optionally includes modifying the first portion of the population of neutrophil cells or the population of neutrophil cells. The modifying can include gene editing. At process block 306, the method 300 includes administering the first portion of the population of neutrophil cells, if the cells have been sorted, or the population of neutrophil cells, if the cells have not been sorted, to the subject.

The neutrophil cells can be harvested from the subject to which they are administered prior to sorting. Blood is collected from a healthy human donor. Neutrophils are isolated from the blood, for example, by magnetic separation. The following measurements of the neutrophils are made in parallel: (i) optical metabolic imaging; metabolomics, for example by LC/MS; and functional measurement, for example, oxidative burst and NETosis, the activation and release of networks of extracellular fibers such as DNA. The neutrophil cells can be either directly introduced to the subject or can undergo additional processing prior to introduction to the subject.

Cellpose is a deep-learning network for segmentation of cells. Dice similarity coefficient (DSC) is used here to evaluate the image segmentation quality by the retrained Cellpose model. Ground truth is defined as the accuracy of a training set's classification for supervised learning techniques, and the DSC gauges the similarity of two samples. Two samples were compared: ground truth (hand segmented cell mask) and the Cellpose generated mask. Cellpose was re-trained using a batch of 241 images (approximately 20384 cells) which resulted in a DSC of 0.909963147795801. The retrained Cellpose model was then tested on a batch of 351 images (32198 cells, with ground truth) and a batch of images (8289 cells, without ground truth) resulting in a DSC of 0.8699530698416. The Cellpose mask representing the Cellpose segmentation results, and ground truth mask, representing the manual segmentation, are compared to NAD(P)H intensity below.

The methods described herein provided surprising results to the inventors. First, it was unclear if the acquired fluorescence data would be capable at all of classifying current neutrophil activation status. While Applicant has previously shown a capability to determine activation state in a different cell type (T cells), it was uncertain whether this capacity would extend to different cell types. In fact, despite both being immune cells, neutrophils and T cells are quite distinct from one another. Neutrophils are part of the innate branch of the immune system while T cells are part of the adaptive branch of the immune system. At the initial level of differentiation from multipotential hematopoietic stem cells, neutrophils emerge from myeloid progenitor cells, whereas T cells differentiate from lymphoid progenitor cells. In other words, at the very first level of distinction between immune cells, these two cell types are different from one another. Second, Applicant attempted similar experimental efforts to classify different categories of T cells but was unable to successfully classify those differences. Previous publication by Yakimov et al measured NAD(P)H fluorescence lifetime in neutrophils (Yakimov B P et al., Biomed Opt Express 2019, PMID: 31453006; doi:10.1364/BOE.10.004220), however they did not measure metabolic changes upon activation. In another publication by Leben et al., phasor analysis of NAD(P)H lifetime was used to show monocyte activation (Leben R et al., Int J Mol Sci 2018, PMID: 29596303, doi: 10.3390/ijms19041018). In this case, significant overlap between unstimulated and activated groups was observed. This shows NAD(P)H phasor analysis alone would not be capable of high classification accuracy for neutrophil activation. Applicant was never of the opinion that the technique would have universal capability and sought specific protection for specific categorization of a specific cell type for the foundational work in this case, which is evidence that the inventors viewed the likelihood of success in categorizing different cell types was very low. Third, Applicant was surprised by the specific metabolic endpoints and morphological parameters that led to satisfactory categorization. In some cases, the systems and methods described herein can provide meaningful classification with only NAD(P)H autofluorescence measurements. While the classification is superior when FAD autofluorescence data is included, Applicant unexpectedly discovered that a satisfactory classification was achieved without requiring data from both chromophores. Applicant emphasizes that other combinations of endpoints are capable of classifying activation status and does not intent to limit the scope of this invention to this one feature.

### Systems

This disclosure also provides systems. The systems can be suitable for use with the methods described herein. When a feature of the present disclosure is described with respect to a given system, that feature is also expressly contemplated as being combinable with the other systems and methods described herein, unless the context clearly dictates otherwise.

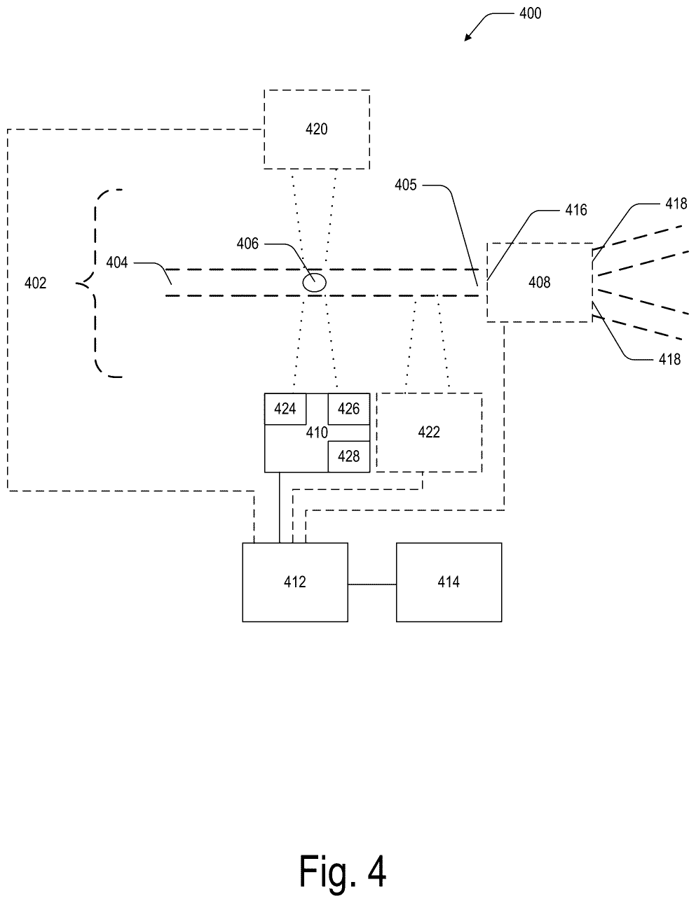

Referring to FIG. 4, the present disclosure provides a neutrophil activation status sensing device 400. The device 400 includes an observation zone 406. The observation zone 406 is adapted to receive a cell analysis pathway 402, a cell culture (not illustrated), or other device or system capable of presenting neutrophil cells for optical interrogation. The device 400 includes a processor 412 and a non-transitory computer-readable medium 414, such as a memory. In some configurations, the processor 412 can be or otherwise include a field-programmable gate array (FPGA). In configurations where the processor 412 is an FPGA, an additional processor (not shown) may be included to capture images.

The device 400 optionally includes a cell analysis pathway 402. The cell analysis pathway 402 includes an inlet 404, the observation zone 406, and an outlet 405. The device 400 optionally includes a cell sorter 408. The observation zone 406 is coupled to the inlet 404 downstream of the inlet 404 and is coupled to the outlet 405 upstream of the outlet 405. The device 400 also includes a single-cell autofluorescence spectrometer 410. The device 400 can further include an optional cell picker (not illustrated).

The inlet 404 can be any nanofluidic, microfluidic, or other cell sorting inlet. A person having ordinary skill in the art of fluidics has knowledge of suitable inlets 404 and the present disclosure is not intended to be bound by one specific implementation of an inlet 404.

The outlet can be any nanofluidic, microfluidic, or other cell sorting outlet. A person having ordinary skill in the art of fluidics has knowledge of suitable outlets 405 and the present disclosure is not intended to be bound by one specific implementation of an outlet 405.

The observation zone 406 is configured to present neutrophil cells for individual autofluorescence decay interrogation. A person having ordinary skill in the art has knowledge of suitable observation zones 406 and the present disclosure is not intended to be bound by one specific implementation of an observation zone 406.

In some cases, the observation zone 406 allows presentations of groups neutrophil cells, such as are present in a subject organism, including mammals, including humans, thereby allowing in vivo determination of neutrophil activation status.

The optional cell sorter 408 has a sorter inlet 416 and at least two sorter outlets 418. The cell sorter is coupled to the observation zone 406 via the sorter inlet 416 downstream of the observation zone 406. The cell sorter 408 is configured to selectively direct a cell from the sorter inlet 416 to one of the at least two sorter outlets 418 based on a sort signal. The sort signal can, for example, originate from the processor and can be based on fluorescence measurements or the current activation prediction.

The inlet 404, observation zone 406, outlet 405, and optional cell sorter 408 can be components known to those having ordinary skill in the art to be useful in high-throughput cell screening devices or flow sorters, including commercial flow sorters. The cell analysis pathway 402 can further optionally include a flow regulator, as would be understood by those having ordinary skill in the art. The flow regulator can be configured to provide flow of cells through the observation zone at a rate that allows the autofluorescence spectrometer 410 to acquire the autofluorescence data set. A useful review of the sorts of fluidics that can be used in combination with the present disclosure is Shields et al., “Microfluidic cell sorting: a review of the advances in the separation of cells from debulking to rare cell isolation,” Lab Chip, 2015 Mar. 7; 15(5): 1230-49, which is incorporated herein by reference in its entirety.

The optional cell picker can serve a similar function as the optional cell sorter 408, namely, isolating cells based on a sort signal. The cell picker can be automated. One example of a suitable cell picker includes an ALS CellCelector™, available commercially from ALS Automated Lab Solutions GmbH, Jena, Germany.

The autofluorescence spectrometer 410 includes a light source 424, a photon-counting detector 426, and photon-counting electronics 428.

The autofluorescence spectrometer 410 can be any spectrometer suitable for acquiring autofluorescence data sets as understood by those having ordinary skill in the optical arts.

Suitable light sources 424 include, but are not limited to, lasers, LEDs, lamps, filtered light, fiber lasers, and the like. The light source 424 can be pulsed, which includes sources that are naturally pulsed and continuous sources that are chopped or otherwise optically modulated with an external component.

The light source 424 can provide pulses of light having a full-width at half maximum (FWHM) pulse width that is of a duration that is adequate to achieve the spectroscopic goals described herein, as would be appreciated by one having ordinary skill in the spectroscopic arts. In some cases, the FWHM pulse width is at least 1 fs, at least 5 fs, at least 10 fs, at least 25 fs, at least 50 fs, at least 100 fs, at least 200 fs, at least 350 fs, at least 500 fs, at least 750 fs, at least 1 ps, at least 3 ps, at least 5 ps, at least 10 ps, at least 20 ps, at least 50 ps, or at least 100 ps. In some cases, the FWHM pulse width is at most 10 ns, at most 1 ns, at most 900 ps, at most 750 ps, at most 600 ps, at most 500 ps, at most 400 ps, at most 250 ps, at most 175 ps, at most 100 ps, at most 75 ps, at most 60 ps, at most 50 ps, at most 35 ps, at most 25 ps, at most 20 ps, at most 15 ps, at most 10 ps, or at most 1 ps.

The light source 424 can emit wavelengths that are tuned to the absorption of NAD(P)H and/or FAD. In some cases, the wavelength is at least 340 nm, at least 345 nm, at least 350 nm, at least 355 nm, at least 360 nm, at least 365 nm, or at least 370 nm. In some cases, the wavelength is at most 415 nm, at most 410 nm, at most 405 nm, at most 400 nm, at most 395 nm, at most 390 nm, at most 385 nm, or at most 380 nm. In some cases, the wavelength is between 360 nm and 415 nm, between 350 nm and 410 nm, or between 370 nm and 380 nm. In some cases, the wavelength is 375 nm. In some cases, the wavelength is 2 times or 3 times these wavelength values (i.e., the frequency is ½ or ⅓). It should be appreciated that pulsed light sources inherently have some degree of bandwidth, so they are never exactly monochromatic. Thus, references herein to “wavelength” refer to either a wavelength at the peak intensity or a weighted average wavelength. In some cases, the pulsed light source 424 is a UV pulsed diode laser. In some cases, the pulsed light source has a wavelength that is double the peak absorption wavelength of NAD(P)H and/or FAD, with an ultrashort pulse duration, such that fluorescence excitation is achieved through two-photon excitation events, as understood by those having ordinary skill in the optical arts.

The photon-counting detector 426 can be any detector suitably capable of detecting single photons and delivering an analog or digital output representative of the detected photons. Examples of photon-counting detectors 426 include, but are not limited to, a photomultiplier tube, a photodiode, an avalanche photodiode, a single-photon avalanche diode (SPAD), a charge-coupled device, combinations thereof, and the like.

The photon-counting electronic 428 can include electronics understood by those having ordinary skill in the art to be suitable for use with single-photon detectors 426 to produce the data sets described herein. Examples of suitable photon-counting electronics 428 include, but are not limited to, a field-programmable gate array (FPGA), a dedicated digital signal processor (DSP) with a digitizer and a time-to-digital converter, a time-correlated single photon counting (TCSPC) electronic board with time-to-amplitude and analog-to-digital converter electronics (as implemented by Becker & Hickl, Berlin, Germany), combinations thereof, and the like.

The autofluorescence spectrometer 410 can be directly (i.e., the processor 412 communicates directly with the spectrometer 410 and receives the signals) or indirectly (i.e., the processor 412 communicates with a sub-controller that is specific to the spectrometer 410 and the signals from the spectrometer 410 can be modified or unmodified before sending to the processor 412) controlled by the processor 412. Autofluorescence data sets can be acquired by known spectroscopic methods. Fluorescence lifetime images can also be acquired by known imaging methods and those acquired images can be used by the systems and methods described herein, as would be understood by those having ordinary skill in the spectroscopic arts. The device 400 can include various optical filters tuned to isolate autofluorescence signals of interest. The optical filters can be tuned to the autofluorescence wavelengths of NAD(P)H and/or FAD.

The autofluorescence spectrometer 410 can be configured to acquire the autofluorescence dataset from the detector's 426 electrical output at a repetition rate understood by those having ordinary skill in the spectroscopic arts to be suitable for providing adequate sampling to observe the dynamics disclosed herein. In some cases, the repetition rate can be at least 1 kHz, at least 5 kHz, at least 10 kHz, at least 30 kHz, at least 50 kHz, at least 100 kHz, at least 500 kHz, at least 750 kHz, at least 1 MHZ, at least 4 MHZ, at least 7 MHz, at least 10 MHZ, at least 15 MHZ, at least 20 MHZ, at least 50 MHz, at least 100 MHz, at least 500 MHz, or at least 1 GHz. In some cases, the repetition rate can be at most 1 THz, at most 800 GHz, at most 500 GHz, at most 250 GHz, at most 150 GHz, at most 100 GHz, at most 70 GHz, at most 50 GHz, at most 25 GHz, at most 15 GHZ, at most 10 GHz, at most 6 GHZ, at most 2 GHz, at most 1 GHz, at most 750 MHZ, at most 500 MHZ, at most 400 MHz, at most 250 MHz, at most 175 MHz, or at most 100 MHz. While there can be downside associated with oversampling, in principle the present disclosure can function with as high of a sampling rate as can be achieved with existing technology. The repetition rates identified herein are based on the state of the art at the time the present disclosure was prepared and filed and are not intended to be limiting in the event that future developments facilitate a greater repetition rate.

The pulsed light source 424 can be configured to operate at pulse repetition rates that are adapted to acquire the needed fluorescence lifetime information. The maximum pulse repetition rate is limited by the fluorescence lifetime of the fluorophore of interest. The fluorescence decay must have fully died down by the time the next pulse of light is introduced to the sample in order to avoid ambiguity about the sources of data sets (i.e., was this particular fluorescent photon initiated by the most recent excitation pulse of light or the one preceding it?). The pulsed light source 424 can have a pulse repetition rate of up to 100 MHz, up to 80 MHz, up to 60 MHz, or up to 40 MHz. The lower limit of the pulse repetition rate is more practical in a sense of reducing the overall sampling time, but theoretically the data can be taken very slowly if there is some reason to do so.

The device 400 can optionally include an optical microscope 420 for acquiring visual images of cells that are located in the observation zone 406 or elsewhere along the cell analysis pathway 402.

The device 400 can optionally include a cell size measurement tool 422. The cell size measurement tool 422 can be any device capable of measuring the size of cells, including but not limited to, an optical microscope, such as optical microscope 420. In some cases, the optical microscope and the cell size measurement tool 422 are the same subsystem.

In some cases, the autofluorescence spectrometer 410 and the optical microscope 420 can be integrated into a single optical subsystem. In some cases, the autofluorescence spectrometer 410 and the cell size measurement tool 422 can be integrated into a single optical subsystem. While some aspects of the methods described herein can operate by not utilizing the cell size as an input to the convolutional neural network, it may be useful to measure the cell size for other purposes.

The processor 412 is in electronic communication with the spectrometer 410. The processor 412 is also in electronic communication with, when present, the optional cell sorter 408, the optional optical microscope 420, and the optional cell size measurement tool 422.

The non-transitory computer-readable medium 414 has stored thereon instructions that, when executed by the processor, cause the processor to execute at least a portion of the methods described herein. Equations for which the first and second phasor coordinates are inputs can also be stored on the non-transitory computer-readable medium 414. The non-transitory computer-readable medium 414 can be local to the device 400 or can be remote from the device, so long as it is accessible by the processor 412.

The device 400 can be substantially free of fluorescent labels (i.e., the cell analysis pathway 402 does not include a region for mixing the cell(s) with a fluorescent label). The device 400 can be substantially free of immobilizing agents for binding and immobilizing neutrophil cells.

### Example 1

Neutrophils were isolated from freshly collected healthy human donor using the MACSxpress Whole Blood Neutrophil Isolation Kit (Miltenyi Biotec 130-104-434) according to the manufacturer's instructions. Each measurement was repeated from at least 5 donors. For activation, neutrophils were treated with 100 nM PMA for 15 mins. Neutrophils were seeded in an imaging dish coated with Poly-D-Lysine.

Two-photon FLIM was performed on a custom-built Ultima Multiphoton Imaging System (Bruker) consisting of an inverted microscope (τ₁-E, Nikon) coupled to an ultrafast tunable laser source (Insight DS+, Spectra Physics). The data was acquired using time correlated single photon counting electronics (SPC 150, Becker & Hickl GmbH). Excitations of 750 nm and 890 nm were used for NAD(P)H and FAD respectively. The samples were illuminated through a 40× water immersion, 1.15 N.A objective (Plan Apo, Nikon) with an image scan speed of 4.8 μs/pixel, 60 seconds integration time, image size of either 256×256 pixels, with 2× digital zoom. Power at the sample was 4-5 mW. A dichroic at 720 nm was used to separate excitation from emission signals. The emission filter used for NAD(P)H and FAD were bandpass 460/80 nm and 500/100 nm respectively. GaAsP photomultiplier tubes (H7422P-40, Hamamatsu, Japan) were used for detection.

For FLIM analysis, fluorescence decay was fit with a double exponential using SPCImage software (Becker & Hickl GmbH). For manual single cell segmentation, a customized CellProfiler pipeline was used. The generated masks served as training data automated segmentation. Automated single cell segmentation was performed using the open-source Python package Cellpose. A stock whole cell U-Net model (‘cyto’) was retrained for 500 epochs on 242 hand segmented masks from varying conditions prior to segmentation of the 219 images within the test dataset.

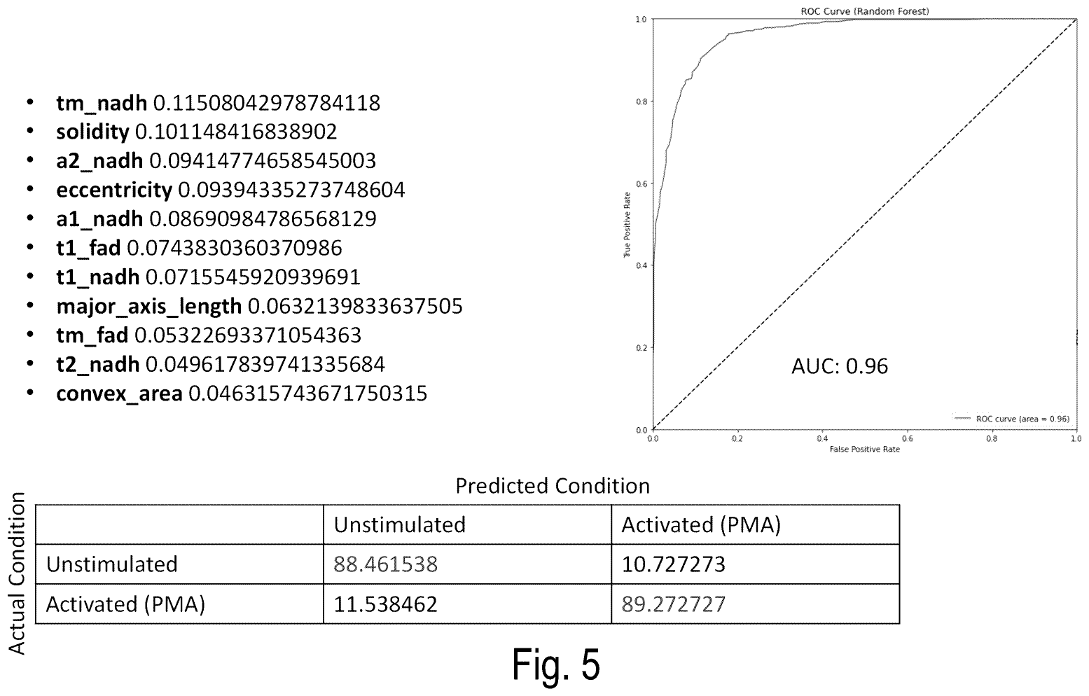

In another instance, using the NAD(P)H τₘ, NAD(P)H α₁, NAD(P)H α₂, NAD(P)H τ₁, NAD(P)H τ₂, FAD τ₁, and FAD τₘ as metabolic endpoints and solidity, eccentricity, and major axis length, and convex area as morphological parameters gave the results shown in FIG. 5, where the morphological parameters were computed from the NADH image.

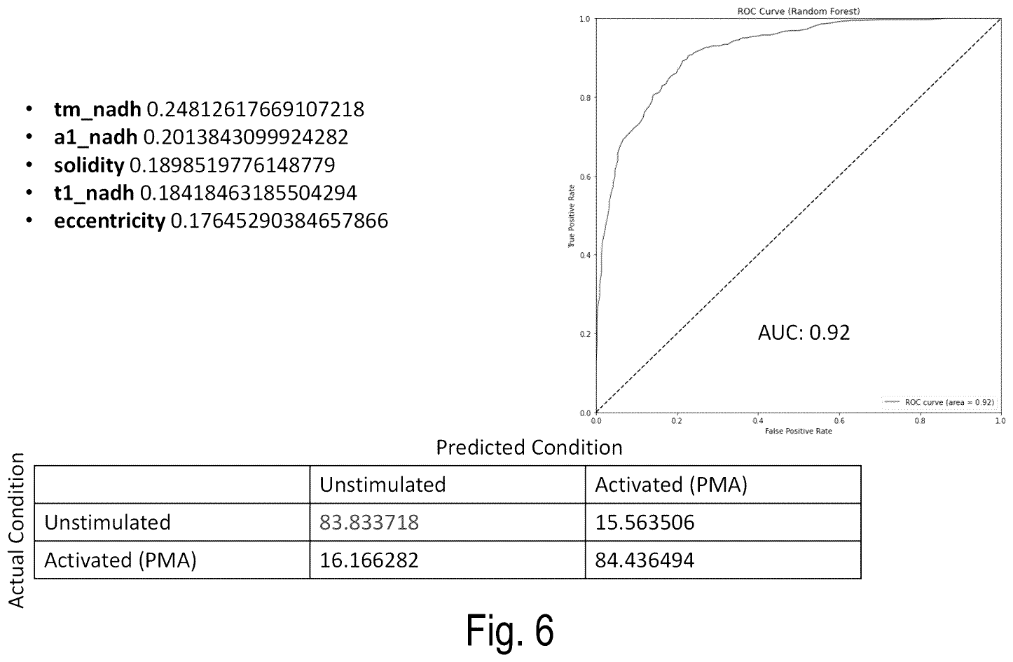

In another instance, using the NAD(P)H τₘ, NAD(P)H α₁, and NAD(P)H shortest fluorescence lifetime component (τ₁) as metabolic endpoints along with solidity and eccentricity as morphological parameters gave the results shown in FIG. 6, where the morphological parameters were computed from the NADH image.

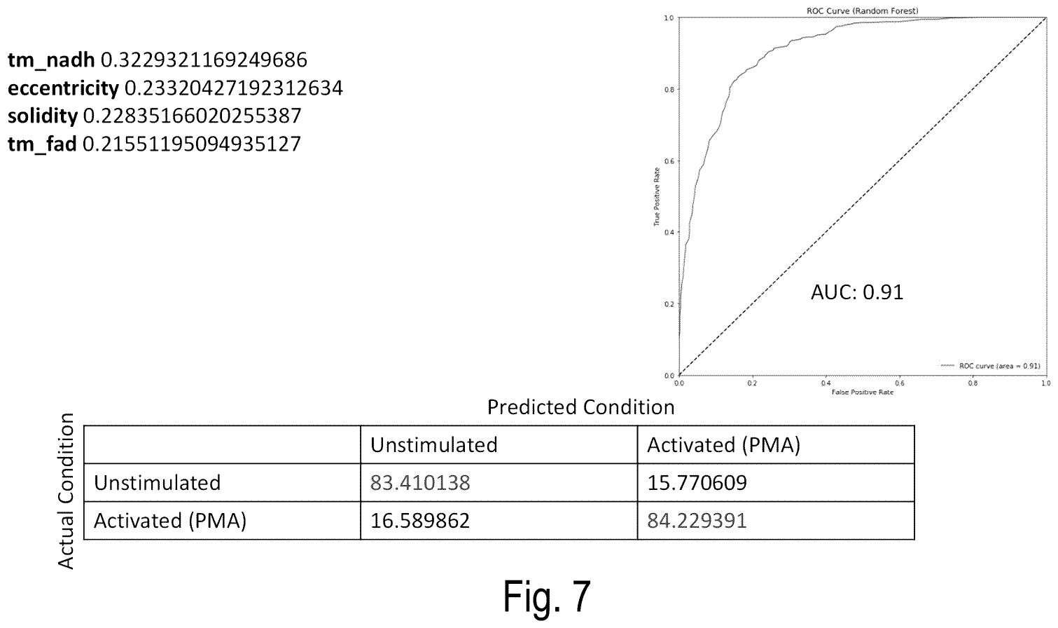

In another instance, using the NAD(P)H τₘ and the FAD τₘ as metabolic endpoints along with solidity and eccentricity as morphological parameters gave the results shown in FIG. 7, where the morphological parameters were computed from the NADH image.

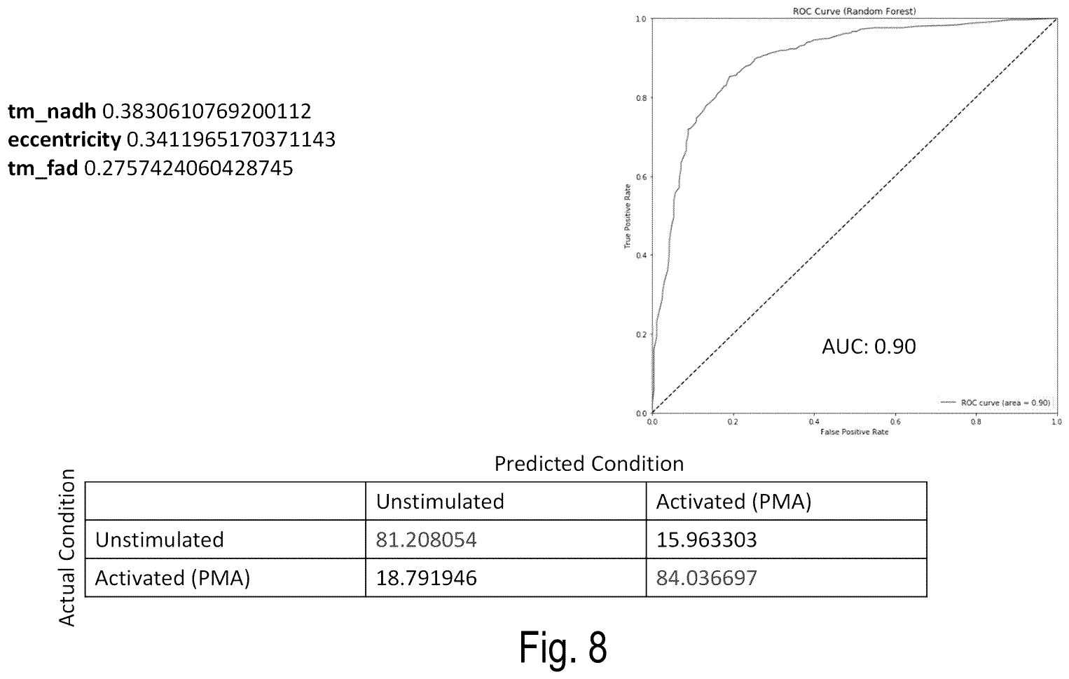

In one instance, using the NAD(P)H τₘ and the FAD τₘ as metabolic endpoints and eccentricity as a morphological parameter gave the results shown in FIG. 8, where the morphological parameter was computed from the NADH image.

### Example 2

Animal care and use was approved by the Institutional Animal Care and Use Committee of University of Wisconsin and strictly followed guidelines set by the federal Health Research Extension Act and the Public Health Service Policy on the Humane Care and Use of Laboratory Animal, administered by the National Institute of Health Office of Laboratory Animal Welfare. All protocols using zebrafish in this study have been approved by the University of Wisconsin-Madison Research Animals Resource Center.

All protocols using zebrafish in this study have been approved by the University of Wisconsin-Madison Research Animals Resource Center (protocols M005405-A02). Adult zebrafish were maintained on a 14 hr:10 hr light/dark schedule. Upon fertilization, embryos were transferred into E3 media (5 mM NaCl, 0.17 mM KCl, 0.44 mM CaCl₂, 0.33 mM MgSO₄, 0.025 mM NaOH, 0.0003% Methylene Blue) and maintained at 28.5° C. Adult transgenic line Tg(mpx:mCherry) was used that labels neutrophils by driving the cytoplasmic expression of mCherry under the myeloid-specific peroxidase promoter (Yoo S K et al., Dev Cell 2010, PMID: 20159593; doi: 10.1016/j.devcel.2009.11.015, which is incorporated herein by reference in its entirety). The transgenic line was outcrossed to casper fish (White R M et al., Cell Stem Cell. 2008 Feb. 7; 2(2): 183-9. PMCID: PMC2292119 PMID: 18371439, doi: 10.1016/j.stem.2007.11.002, which is incorporated herein by reference in its entirety) to generate transgenic line devoid of pigmentation.

Work by others has shown that transgenic zebrafish lines with fluorescently labeled leukocytes have made it possible to visualize the neutrophil response in real time by use of optically transparent zebrafish larvae. (Harvie E A, Huttenlocher A., J Leukoc Biol. 2015 October; 98(4):523-37.doi: 10.1189/jlb.4MR1114-524R. Epub 2015 Feb. 25. PMID: 25717145; PMCID: PMC4569048, which is incorporated herein by reference in its entirety.) For live in vivo imaging, the larvae were loaded in a zWEDGI device (Huemer K et al., PMID: 27676647, doi: 10.1089/zeb.2016.1323, which is incorporated herein by reference in its entirety.) in E3 media without methylene blue and supplemented with 0.2 mg/mL Tricaine (ethyl 3-aminobenzoate; Sigma-Aldrich) to anesthetize larvae. The head region was secured by adding 2% low gelling agarose (A9045, Sigma) to prevent drifting during imaging.

Two-photon FLIM was performed on a custom-built Ultima Multiphoton Imaging System (Bruker) consisting of an inverted microscope (TI-E, Nikon) coupled to an ultrafast tunable laser source (Insight DS+, Spectra Physics). The data was acquired using time correlated single photon counting electronics (SPC 150, Becker & Hickl GmbH). The emission was collected using bandpass filters of 466/40 nm (NAD(P)H), 514/30 nm (FAD), and 590/45 nm (mCherry) prior to detection with GaAsP photomultiplier tubes (Hamamatsu). All three fluorophores were concurrently excited using a previously reported wavelength mixing approach (Mahou et al., 2012; Stringari et al., 2017). Briefly, the laser source tuned to 750 nm (NAD(P)H excitation) was delayed and collimated with the secondary laser line fixed at 1,041 nm (mCherry excitation) for spatial and temporal overlap at each raster-scanned focal point (2-color excitation of FAD with 750 nm+1041 nm). The samples were illuminated through a 40× water immersion, 1.15 N.A objective (Plan Apo, Nikon) with an image scan speed of 4.8 μs/pixel, 60 seconds integration time, image size of either 256×256 pixels, with 3× digital zoom. Power at the sample was 4-5 mW.

For FLIM analysis, fluorescence decay was fit with a double exponential using SPCImage software (Becker & Hickl GmbH). mCherry intensity image was used for automated single cell segmentation of neutrophils using Cellpose.

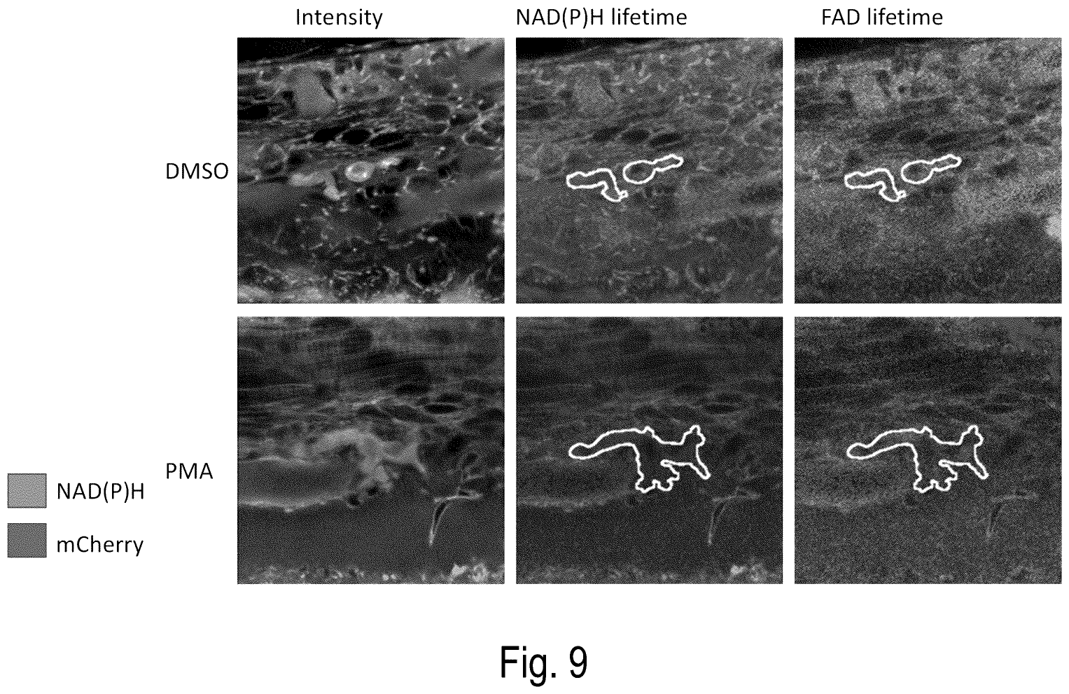

Results of the in vivo metabolic imaging study based on NAD(P)H lifetime and FAD lifetime as described above are shown in FIG. 9, which demonstrates the regions in the image corresponding to neutrophil activation with DMSO or PMA. The neutrophil activation is confirmed by visualizing mCherry.

We claim:

1. A neutrophil activation status sensing device comprising:
a cell analysis observation zone adapted to receive a neutrophil cell and to present the neutrophil cell for individual autofluorescence interrogation;
an autofluorescence spectrometer configured to acquire an autofluorescence data set for the neutrophil cell located in the cell analysis observation zone, the autofluorescence spectrometer comprising a light source, a photon-counting detector, and photon-counting electronics;
a processor in electronic communication with the autofluorescence spectrometer; and
a non-transitory computer-readable medium accessible to the processor and having stored thereon instructions that, when executed by the processor, cause the processor to:
a) receive the autofluorescence data set; and
b) identify a current activation status of the neutrophil cell based on a current activation prediction, wherein the current activation prediction is computed using at least a portion of the autofluorescence data set, wherein the current activation prediction is computed using at least two metabolic endpoints of the autofluorescence data set and at least one morphological parameter as an input,
wherein the at least two metabolic endpoints include either:
reduced nicotinamide adenine dinucleotide and/or reduced nicotinamide dinucleotide phosphate (NAD(P)H) mean fluorescence lifetime (τₘ), NAD(P)H shortest fluorescence amplitude component (α₁), and NAD(P)H shortest fluorescence lifetime component (τ₁); or
the NAD(P)H τₘ and flavin adenine dinucleotide (FAD) τₘ,
wherein the at least one morphological parameter includes either solidity or eccentricity.

2. The neutrophil activation status sensing device of claim 1, the device further comprising a cell analysis platform adapted to receive a cell culture containing the neutrophil cell of interest, the cell analysis platform adapted to position the cell culture containing the neutrophil cell of interest in the observation zone.

3. The neutrophil activation status sensing device of claim 1, the device further comprising a cell analysis pathway comprising: (i) an inlet; (ii) the observation zone coupled to the inlet downstream of the inlet, the observation zone configured to present neutrophil cells for individual autofluorescence interrogation; and (iii) an outlet coupled to the observation zone downstream of the observation zone.

4. The neutrophil activation status sensing device of claim 3, the neutrophil activation status sensing device further comprising a flow regulator coupled to the inlet.

5. The neutrophil activation status sensing device of claim 4, wherein the flow regulator is configured to provide flow of cells through the observation zone at a rate that allows the autofluorescence spectrometer to acquire the autofluorescence data set for the neutrophil cell when it is positioned in the observation zone.

6. The neutrophil activation status sensing device of claim 3, wherein the cell analysis pathway does not include a fluorescent label for binding to the neutrophil cell.

7. The neutrophil activation status sensing device of claim 3, wherein the cell analysis pathway does not include an immobilization agent for binding and immobilizing neutrophil cells.

8. The neutrophil activation status sensing device of claim 3, the neutrophil activation status sensing device further comprising a cell sorter having a sorter inlet and at least two sorter outlets, the cell sorter coupled to the cell analysis pathway via the outlet downstream of the observation zone, the cell sorter configured to selectively direct a cell from the sorter inlet to one of the at least two sorter outlets based on a sort signal, the processor in electronic communication with the cell sorter, and the instructions, when executed by the processor, further cause the processor to provide the sort signal to the cell sorter based on the current activation prediction.

9. The neutrophil activation status sensing device of claim 1, wherein the cell analysis observation zone is adapted to receive a live subject for the purpose of in vivo determination of activation status for neutrophils of interest within the subject.

10. The neutrophil activation status sensing device of claim 1, wherein the subject is a mammalian subject, optionally a human subject.

11. The neutrophil activation status sensing device of claim 1, the autofluorescence spectrometer comprising a detector-side filter configured to transmit fluorescence signals of interest.

12. The neutrophil activation status sensing device of claim 1, the neutrophil activation status sensing device further comprising at least one of a cell size measurement tool configured to measure cell size and to communicate the cell size to the processor and a cell imager configured to acquire an image of a cell positioned within the observation zone and to communicate the image to the processor.

13. The neutrophil activation status sensing device of claim 1, wherein the instructions, when executed by the processor, further cause the processor to generate a report including the current activation prediction for neutrophil cells analyzed by the device.

14. A method of characterizing neutrophil activation status, the method comprising:
a) optionally receiving a population of neutrophil cells having unknown activation status;
b) acquiring an autofluorescence data set from a neutrophil cell of the population of neutrophil cells; and
c) identifying a current activation status of the neutrophil cell based on a current activation prediction, wherein the current activation prediction is computed using at least a portion of the autofluorescence data set, wherein the current activation prediction is computed using at least two metabolic endpoints of the autofluorescence data set and at least one morphological parameter as an input,
wherein the at least two metabolic endpoints include either:
reduced nicotinamide adenine dinucleotide and/or reduced nicotinamide dinucleotide phosphate (NAD(P)H) mean fluorescence lifetime (τₘ), NAD(P)H shortest fluorescence amplitude component (α₁), and NAD(P)H shortest fluorescence lifetime component (τ₁);
the NAD(P)H τₘ and flavin adenine dinucleotide (FAD) τₘ,
wherein the at least one morphological parameter includes either solidity or eccentricity.

15. The method of claim 14, wherein the population of neutrophil cells are located in a subject and the method is an in vivo method.

16. A method of classifying neutrophil activation status, the method comprising:
a) receiving a population of neutrophil cells having unknown activation status;
b) acquiring an autofluorescence data set for each neutrophil cell of the population of neutrophil cells, each autofluorescence data set including autofluorescence lifetime information; and
either:
c1) physically isolating a first portion of the population of neutrophil cells from a second portion of the population of neutrophil cells based on a current activation prediction, wherein each neutrophil cell of the population of neutrophil cells is placed into the first portion when the current activation prediction exceeds a predetermined threshold and into the second portion when the current activation prediction is less than or equal to the predetermined threshold; or
c2) generating a report including the current activation prediction, the report optionally identifying a proportion of the population of neutrophil cells having the current activation prediction that exceeds the predetermined threshold,
wherein the current activation prediction is computed using at least two metabolic endpoints of the autofluorescence data set and at least one morphological parameter as an input, wherein the at least two metabolic endpoints include either:
reduced nicotinamide adenine dinucleotide and/or reduced nicotinamide dinucleotide phosphate (NAD(P)H) mean fluorescence lifetime (τₘ), NAD(P)H shortest fluorescence amplitude component (α₁), and NAD(P)H shortest fluorescence lifetime component (τ₁); or
the NAD(P)H τₘ and flavin adenine dinucleotide (FAD) τₘ,
wherein the at least one morphological parameter includes either solidity or eccentricity.

17. The method of claim 16, wherein the method does not involve use of a fluorescent label for binding to the neutrophil cell.

18. The method of claim 16, wherein the method does not involve immobilizing the neutrophil cell.

19. A method of administering activated neutrophil cells to a subject in need thereof, the method comprising:
e) the method of claim 14, wherein the method comprises step c2); and
f) in response to the proportion of neutrophil cells having the current activation prediction exceeding a second predetermined threshold, introducing the population of neutrophil cells to the subject.

20. The method of claim 19, wherein the population of neutrophil cells is modified prior to step f).
